(19) United States
(12) Patent Application Publication
(10) Pub. No.: US 2023/0355944 A1
(43) Pub. Date: Nov. 9, 2023

(54) Split Septum Needle Free Connector with Improved Flushing Features for Macrobore Side Port

(71) Applicant: Becton, Dickinson and Company, Franklin Lakes, NJ (US)

(72) Inventors: Erica E. Neumann, Sandy, UT (US); Jonathan Karl Burkholz, Salt Lake City, UT (US)

(21) Appl. No.: 18/144,511

(22) Filed: May 8, 2023

Related U.S. Application Data
(60) Provisional application No. 63/339,776, filed on May 9, 2022.

(51) Int. Cl. A61M 39/10; A61M 39/22; A61B 5/15

(52) U.S. Cl. CPC A61M 39/10; A61M 39/22; A61B 5/150992

(57) ABSTRACT

An integrated intravenous catheter system including a catheter adapter having a catheter and an inlet, as well as a needle free connector. The needle free connector includes a proximal port, a distal port positioned opposite the proximal port, and a side port positioned between the proximal port and the distal port. The system also includes extension tubing extending from the side port. The side port includes a tube receiving portion having a first inner diameter sized and configured to receive a distal portion of the extension tubing, an inlet portion having a second inner diameter smaller than the first inner diameter, and a tapered portion extending between the tube receiving portion and the inlet portion.

### CROSS-REFERENCE TO RELATED APPLICATION

The present application claims priority to U.S. Provisional Application Ser. No. 63/339,776, entitled “Split Septum Needle Free Connector with Improved Flushing Features for Macrobore Side Port”, filed May 9, 2022, the entire disclosure of which is hereby incorporated by reference in its entirety.

### BACKGROUND OF THE INVENTION

### Field of the Invention

The present disclosure relates to an integrated intravenous catheter with a needle free connector (NFC) configured for use with a blood draw device.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (“PIVC”), such as, e.g., the BD NEXIVA™ Closed IV Catheter system from Becton, Dickinson and Co. The over-the-needle catheter may be mounted over an introducer needle having a sharp distal tip. The catheter and the introducer needle may be assembled so that the distal tip of the introducer needle extends beyond the distal tip of the catheter with the bevel of the needle facing up away from a skin surface of the patient. The catheter and introducer needle are generally inserted at a shallow angle through the skin into the vasculature of the patient. In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Blood withdrawal using a peripheral IV catheter may be difficult for several reasons, particularly when an indwelling time of the catheter is more than one day. For example, when the catheter is left inserted in the patient for a prolonged period of time, the catheter or vein may be more susceptible to narrowing, collapse, kinking, blockage by debris (e.g., fibrin or platelet clots), and adhering of a tip of the catheter to the vasculature. Due to this, catheters may often be used for acquiring a blood sample at a time of catheter placement but are much less frequently used for acquiring a blood sample during the catheter dwell period.

Accordingly, blood draw devices have been developed to collect blood samples through an existing PIVC. Blood draw devices attach to the PIVC and include a flexible flow tube that is advanced through the PIVC, beyond the catheter tip, and into a vessel to collect a blood sample. After blood collection, the blood draw device is removed from the PIVC and discarded. One example of such a blood draw device, known as PIVO™ from Becton, Dickinson and Company, is shown and described in, e.g., U.S. Pat. No. 11,090,461, which is hereby incorporated by reference in its entirety. As described in U.S. Pat. No. 11,090,461, the blood draw device includes an introducer having an actuator slidably coupled thereto, with the actuator being configured to selectively advance the flexible flow tube through the PIVC. The introducer is couplable to, e.g., a proximal port of a needle free connector (NFC), with the NFC configured to receive a connector or lock positioned on a distal end portion of the introducer.

In addition to the proximal port, the NFC may also include a side port, with the side port couplable to extension tubing used for the introduction of, e.g., a flushing fluid into the NFC. The dimensions of the extension tubing and/or the side port may be chosen with respect to the gauge and/or other dimensions of the indwelling catheter to which the NFC is coupled. For example, referring to FIG. 1, a NFC 100 in accordance with an embodiment of the prior art is shown, with NFC 100 being configured for use with, e.g., 22G and 24G PIVCs. The NFC 100 includes a proximal port 102 having a split septum valve 103 at a proximal end thereof, a side port 104, and a distal end portion configured to receive a short extension tube 105, with the short extension tube 105 configured to be couplable to a catheter adapter (not shown). The side port 104 is configured to receive a long extension tube 106, with the long extension tube 106 having a “microbore” inner channel 108. The inner diameter of the inner channel 108 is relatively small, resulting in a relatively high fluid velocity of a flushing fluid delivered through the long extension tube 106.

The NFC 100 may further include an internal structure 111 configured to create a vortex or otherwise redirect fluid when fluid enters the NFC 100 via the side port 104. In some embodiments, the vortex-creating feature of the internal structure 111 is the same or similar to the flushing features shown and described in U.S. Patent Application Publication No. 2021/0220548, which is hereby incorporated by reference in its entirety. The relatively high velocity of fluid flow through the “microbore” inner channel 108, coupled with the vortex-creating feature of internal structure 111, may result in a flushing of the main channel 110 of NFC 100 that removes a substantial portion of the volume fraction of blood 112 remaining in the NFC 100 after, e.g., a 5 mL flush volume.

Referring to FIG. 2, a NFC 150 in accordance with another embodiment of the prior art is shown, with NFC 150 being configured for use with larger gauge PIVCs such as, e.g., 18G and 20G PIVCs. The NFC 150 includes a proximal port 152 having a split septum valve 153 at a proximal end thereof, a side port 154, and a distal end portion configured to receive a short extension tube 155 couplable to a catheter adapter (not shown). The side port 154 is configured to receive a long extension tube 156. However, unlike long extension tube 106 described above with respect to FIG. 1, with the long extension tube 156 has a “macrobore” inner channel 158 having a relatively large inner diameter. This “macrobore” configuration results in increased gravity flow and power injection flow rate performance of a flushing procedure, but also reduces the fluid velocity of a flushing fluid delivered through the long extension tube 156 and into the main channel 160 of the NFC 150. Even with the presence of a vortex-creating feature such as internal structure 161 within the main channel 160, this reduction in fluid velocity due to the “macrobore” configuration may result in a relatively large volume fraction of blood 162 remaining in the NFC 150 after, e.g., a 5 mL flush volume.

### SUMMARY OF THE INVENTION

Accordingly, there is a need to provide a needle free connector (NFC) configured for use with “macrobore” extension tubing and having improved flushing characteristics.

In accordance with an aspect of the present disclosure, an integrated intravenous catheter system is disclosed. The system may include a catheter adapter having a catheter and an inlet, the catheter configured to be inserted into a patient's vasculature, and a needle free connector including a proximal port, a distal port positioned opposite the proximal port, and a side port positioned between the proximal port and the distal port. The system may also include extension tubing extending from the side port of the needle free connector, wherein the side port of the needle free connector includes a tube receiving portion having a first inner diameter sized and configured to receive a distal portion of the extension tubing, an inlet portion having a second inner diameter smaller than the first inner diameter, and a tapered portion extending between the tube receiving portion and the inlet portion.

In some embodiments, the needle free connector includes a first body portion and a second body portion, wherein the first body portion and the second body portion define a flow path extending between the proximal port and the distal port.

In some embodiments, the inlet portion of the side port fluidly couples the extension tubing to the flow path of the needle free connector.

In some embodiments, the side port is offset from a center of the flow path.

In some embodiments, the second body portion of the needle free connector includes an internal structure configured to redirect fluid when fluid enters the needle free connector via the side port.

In some embodiments, the first body portion and the second body portion of the needle free connector define a longitudinal axis extending between the proximal port and the distal port, and the side port extends from second body portion at an angle of 30-150 degrees relative to the longitudinal axis.

In some embodiments, an interior diameter of the extension tubing is greater than the second inner diameter of the inlet portion of the side port.

In some embodiments, the proximal port of the needle free connector includes a valve member.

In some embodiments, the valve member includes a split septum valve.

In some embodiments, the system further includes a medical connector positioned at a proximal end of the extension tubing.

According to another aspect of the present disclosure, a needle free connector is disclosed, the needle free connector including a first body portion, a second body portion coupled to the first body portion, a proximal port positioned at a proximal end portion of the first body portion, a distal port positioned at a distal end portion of the second body portion, and a side port positioned between the proximal port and the distal port. The side port may include a tube receiving portion having a first inner diameter, an inlet portion having a second inner diameter smaller than the first inner diameter, and a tapered portion extending between the tube receiving portion and the inlet portion.

In some embodiments, the first body portion and the second body portion define a flow path extending between the proximal port and the distal port.

In some embodiments, the side port is offset from a center of the flow path.

In some embodiments, the second body portion includes an internal structure configured to redirect fluid when fluid enters the needle free connector via the side port.

In some embodiments, the first body portion and the second body portion of the needle free connector define a longitudinal axis extending between the proximal port and the distal port, and the side port extends from second body portion at an angle of 30-150 degrees relative to the longitudinal axis.

In some embodiments, the proximal port includes a valve member.

In some embodiments, the valve member includes a split septum valve.

In accordance with another aspect of the present disclosure, a needle free connector is disclosed, the needle free connector including a first body portion, a second body portion coupled to the first body portion, a proximal port positioned at a proximal end portion of the first body portion, a distal port positioned at a distal end portion of the second body portion, wherein the first body portion and the second body portion define a flow path extending between the proximal port and the distal port, and a side port positioned between the proximal port and the distal port. The side port includes a primary channel portion and a tapered portion extending between the primary channel portion and the flow path.

In some embodiments, the side port includes a luer connector on a proximal end thereof.

In some embodiments, the side port includes a secondary needle free connector positioned on a proximal end thereof.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a side cross-sectional view of a needle-free connector coupled to extension tubing having a first inner diameter in accordance with an embodiment of the prior art;

FIG. 2 is a side cross-sectional view of a needle-free connector coupled to extension tubing have a second inner diameter in accordance with another embodiment of the prior art;

FIG. 3 is a perspective view of an integrated intravenous catheter in accordance with an aspect of the present disclosure;

FIG. 4 is a perspective view of a blood draw device configured for use with the integrated intravenous catheter of FIG. 3;

FIG. 5 is a side cross-sectional view of a needle free connector in accordance with an aspect of the present disclosure;

FIG. 6 is a side cross-sectional view of the needle free connector of FIG. 5 coupled to extension tubing and including flushing fluid therein;

FIG. 7 is a side cross-sectional view of a needle free connector in accordance with another aspect of the present disclosure; and

FIG. 8 is a side cross-sectional view of a needle free connector in accordance with another aspect of the present disclosure.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a blood draw device in preparation for or during use. Similarly, in this application, the terms “in the distal direction” and “distally” mean in the direction toward the indwelling catheter, and the terms “in the proximal direction” and “proximally” mean in the direction opposite the direction of the indwelling catheter.

Spatial or directional terms, such as “left”, “right”, “inner”, “outer”, “above”, “below”, and the like, are not to be considered as limiting as the invention can assume various alternative orientations.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

Embodiments of the present disclosure will primarily be described in the context of vascular access systems including an integrated peripheral IV catheter (PIVC). While not shown or described herein, it is to be understood that the connector assemblies described below may be utilized for blood draw and/or probe advancement through any suitable vascular access device such as, e.g., the BD NEXIVA™ Closed IV Catheter system. However, embodiments of the present disclosure equally extend to use with other catheter devices.

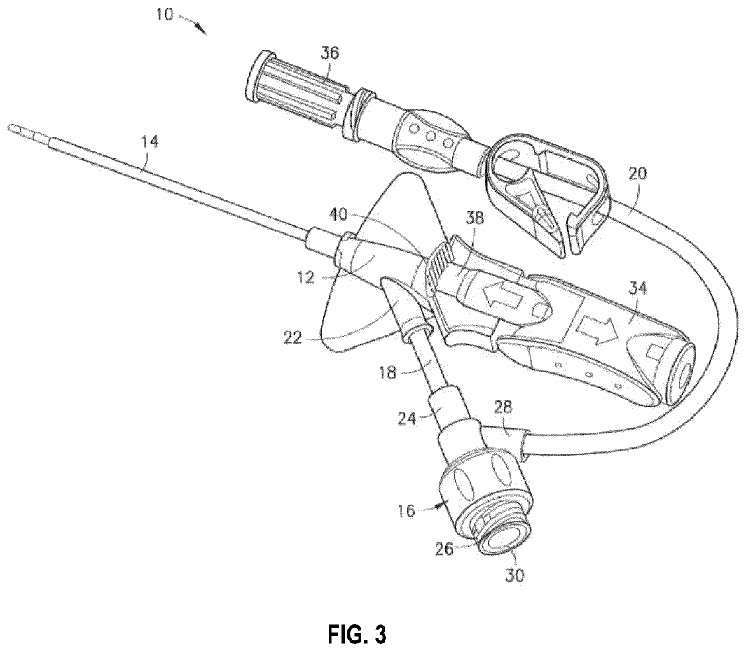

Referring to FIG. 3, an integrated intravenous catheter 10 in accordance with an aspect of the present disclosure is shown. The integrated intravenous catheter 10 includes a catheter adapter 12 having a catheter 14 configured to be inserted into a patient's vasculature, a needle free connector (NFC) 16, an intermediate fluid path 18, and extension tubing 20. The catheter adapter 12 includes an inlet 22. In the embodiment shown in FIG. 3, the needle free connector 16 includes a distal port 24, a proximal port 26 positioned opposite the distal port 24, and a side port 28 positioned between the distal port 24 and the proximal port 26. In some embodiments, the proximal port 26 includes a valve member 30. In some embodiments, the valve member 30 is configured as a split septum valve.

In some embodiments, the intermediate fluid path 18 extends between the inlet 22 of the catheter adapter 12 and the distal port 24 of the needle free connector 16. In some embodiments, intermediate fluid path 18 is formed by a length of tubing. However, intermediate fluid path 18 is not limited to tubing, and may be any appropriate fluid path such as, e.g., a luer connector, etc. Alternatively, in other embodiments, the intermediate fluid path 18 may be omitted, with the needle free connector 16 being coupled directly to the catheter adapter 12. The extension tubing 20 extends from the side port 28 of the needle-free connector 16. The intermediate tubing 18 is configured to provide flexibility when inserting and dressing the catheter 14 and also when manipulating the needle free connector 16 for flushing, blood draw, and/or other procedure without disturbing the catheter insertion site.

Referring still to FIG. 3, in some embodiments, the integrated catheter 10 includes a needle hub assembly 34 and a medical component 36, such as, e.g., a vent plug, with the medical component 36 coupled to the side port 28 of the needle free connector 16 via the extension tubing 20. The needle hub assembly 34 is assembled with the catheter adapter 12 by inserting a needle (not shown) into a lumen of the catheter 14. In one aspect or embodiment, the needle hub assembly 34 includes a needle shield 38 configured to secure a tip of the needle within the needle shield 38 after use. The needle shield 38 may be activated passively. The needle hub assembly 34 may include a push tab 40 to facilitate catheter advancement during insertion. The push tab 40 also allows for one-handed or two-handed advancement. In one aspect or embodiment, the catheter adapter 12 includes one or more wings, as shown, configured to engage a skin surface of a patient. In another aspect or embodiment, the catheter adapter 12 does not include wings.

In some embodiments, at least a portion of the needle free connector 16 is transparent. The connector components of the integrated catheter 10 may be transparent, opaque, and/or colored. In one aspect or embodiment, the needle free connector 16 may include an anti-reflux valve.

In some embodiments, the medical component 36 at the end of the extension tubing 20 is a single port or dual port connector and may include a variety of connectors, including needle free connectors or needle access connectors, such as a PRN. The extension tubing 20 may be left or right facing. In some embodiments, in addition to a vent plug, the medical component 36 may be a removable or non-removable needle free connector or needle access connectors, such as a PRN, that is attached to a female luer connection provided on the extension tubing 20. In some embodiments, a dual female luer port may be bonded or otherwise attached to the extension tubing 20 instead of a single luer connector.

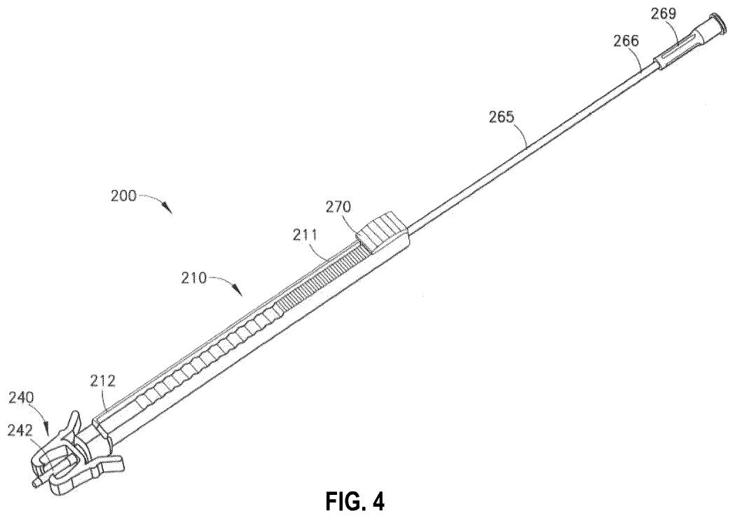

Next, referring to FIG. 4, a blood draw device 200 in accordance with an aspect of the present disclosure is illustrated. The blood draw device 200 may be, e.g., the PIVO™ blood draw device from Becton, Dickinson and Company. In one aspect or embodiment, the blood draw device 200 is the same or similar to the blood draw device shown in U.S. Pat. No. 11,090,461, which is hereby incorporated by reference in its entirety. In one aspect or embodiment, the blood draw device 200 may be any device that advances tubing, a probe, a guidewire, instrument, and/or sensor into the fluid path of the integrated intravenous catheter 10 or beyond the tip of the catheter 14.

Blood draw device 200 may include an introducer 210, a lock 240, a secondary catheter 265, and an actuator 270. The introducer includes a proximal end portion 211 and a distal end portion 212, with the lock 240 being located adjacent the distal end portion 212. The secondary catheter 265 includes the proximal end portion 266 which is coupled to and/or otherwise includes a coupler 269. The coupler 269 is configured to physically and fluidically couple the secondary catheter 265 to any suitable device such as, for example, a fluid reservoir, fluid source, syringe, evacuated container holder (e.g., having a sheathed needle or configured to be coupled to a sheathed needle), pump, and/or the like.

In accordance with some embodiments, a user may manipulate the blood draw device 200 to couple the lock 240 to, e.g., the needle free connector 16. For example, in some embodiments, the user can exert a force sufficient to pivot the first and second clip arms of the lock 240 such that a portion of the needle free connector 16 can be inserted into the space defined between the arms of the lock 240 and, for example, a distal core 242 extending distally from the lock 240. In some embodiments, the distal core 242 can be inserted into, e.g., the proximal port 26 of the needle free connector 16 when the lock 240 is coupled thereto, while the first and second clip arms of the lock 240 may latch onto an exterior surface (or surfaces) of the needle free connector 16 to hold the blood draw device 200 in place relative to the catheter adapter 12. The distal core 242 is sufficiently long to dispose at least a portion of the distal core 242 through the valve member 30 of the needle free connector 16, thereby providing a path for the flow tube or probe to pass from the blood draw device 200 through the catheter adapter 12 of the integrated catheter 10.

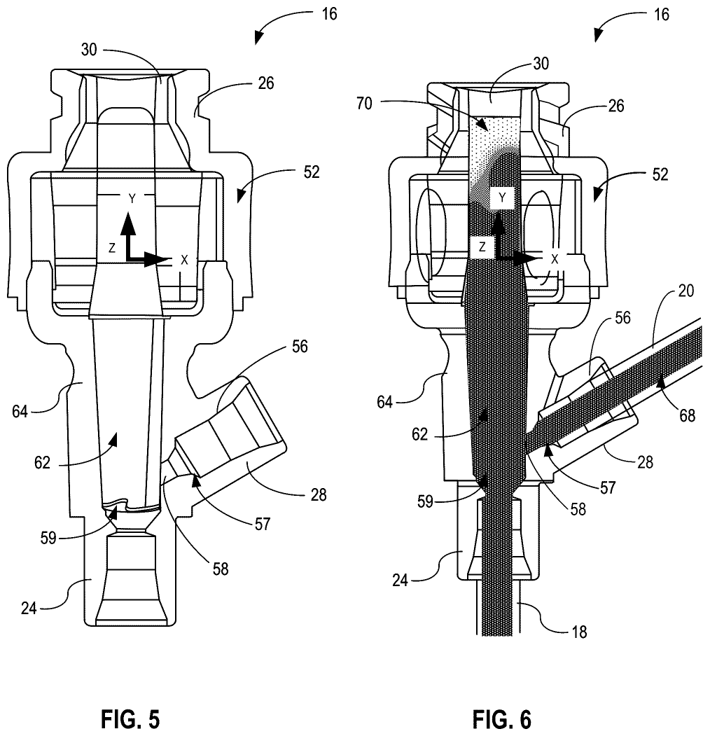

Next, referring to FIGS. 5 and 6, various details of needle free connector 16 in accordance with an aspect of the present disclosure are shown. The needle free connector 16 includes a proximal body 52 and a distal body 64, with the proximal body 52 and the distal body 64 coupled together by any appropriate method. In some embodiments, the proximal body 52 and the distal body 64 may be integrally formed. The proximal body 52 and distal body 64 define a flow path 62 extending between the distal port 24 and the proximal port 26. In some embodiments, the side port 28 may be offset from a center of the flow path 62. Such an offset of the side port 28 may be configured to cause fluid entering the flow path 62 via the side port 28 to enter along an interior surface of the distal body 64 and cause a vortex or otherwise redirect fluid within the distal body 64 and the proximal body 52 to aid flushing of the needle free connector 16. In some embodiments, the distal body 64 of the needle free connector 16 further includes internal structure 59 configured to create a vortex or otherwise redirect fluid when fluid enters the needle free connector 16 via the side port 28. In some embodiments, the offset and vortex-creating feature is the same or similar to the flushing features shown and described in U.S. Patent Application Publication No. 2021/0220548, which is hereby incorporated by reference in its entirety.

The proximal body 52 and the distal body 64 of the needle free connector 16 defines a longitudinal axis extending between the distal port 24 and the proximal port 26, with the side port 28 extending from the distal body 64 at an angle of, e.g., 30°-150° relative to the longitudinal axis of the distal body 64. In one embodiment, the side port 28 extends from the distal body 64 at an angle of 60° relative to the longitudinal axis of the distal body 64.

Referring still to FIGS. 5 and 6, the side port 28 of needle free connector 16 includes a tube receiving portion 56, a tapered portion 57, and an inlet portion 58, wherein inlet portion 58 provides fluid communication to the flow path 62. As is shown in FIG. 6, the tube receiving portion 56 includes an inner diameter sized and configured to securely receive a distal end of the extension tubing 20, with the inner diameter of the tube receiving portion 56 being substantially larger than an inner diameter of the inlet portion 58, with the tapered portion 57 acting to gradually reduce the inner diameter of the side port 28 between the tube receiving portion 56 and the inlet portion 58.

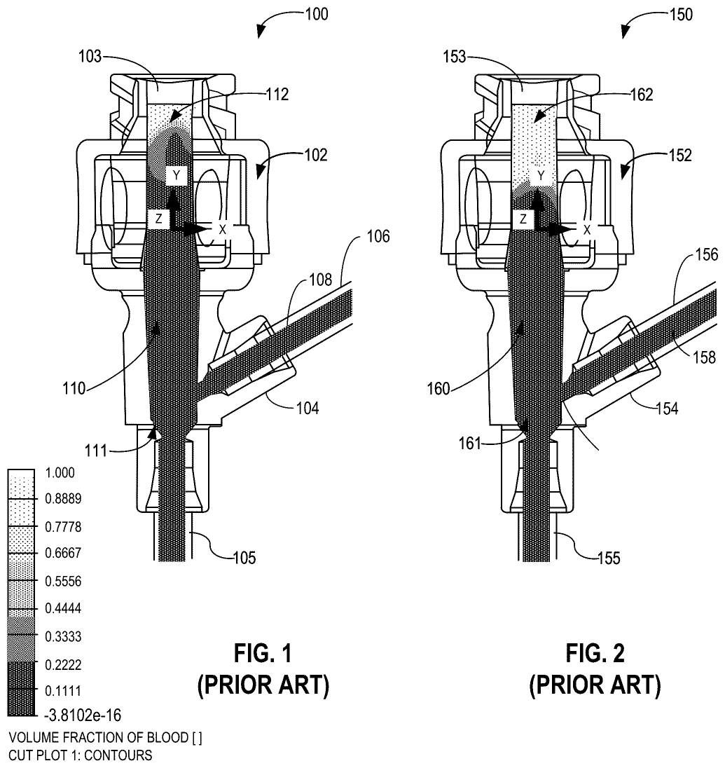

In the embodiment shown in FIG. 6, the extension tubing 20 is configured with a “macrobore” inner channel 68 having a relatively large inner diameter, thereby enabling increased gravity flow and power injection flow rate performance of an infusion procedure. However, unlike the prior art “macrobore” extension tubing and side port described above with respect to FIG. 2, which resulted in decreased fluid velocity and, thus, reduced flushing performance, the combination of the tapered portion 57 and inlet portion 58 acts forms a gradual restriction between the side port 28 and the flow path 62, acting as a nozzle to increase the fluid velocity of the fluid delivered through the extension tubing 20 as it enters the flow path 62. This increased fluid velocity may result in a relatively low volume fraction of blood 70 remaining in the NFC 16 after, e.g., a 5 mL flush volume being delivered through the extension tubing 20, on par with the reduction of volume fraction of blood remaining when “microbore” extension tubing is used, as shown and described with respect to FIG. 1. In some embodiments, the effects of the increased fluid velocity may be coupled with vortex-creating internal structure 59 and/or an offset of side port 28 to further aid in the flushing of blood remaining in the NFC 16.

While not shown in FIG. 6, it is to be understood that small inner diameter of inlet portion 58 also enables NFC 16 to be used with “microbore” extension tubing without the need to alter the body mold of NFC 16 between “macrobore” and “microbore” configurations. Thus, NFC 16 may be used for any suitable catheter gauge such as, e.g., 18G, 20G, 22G, 24G, etc.

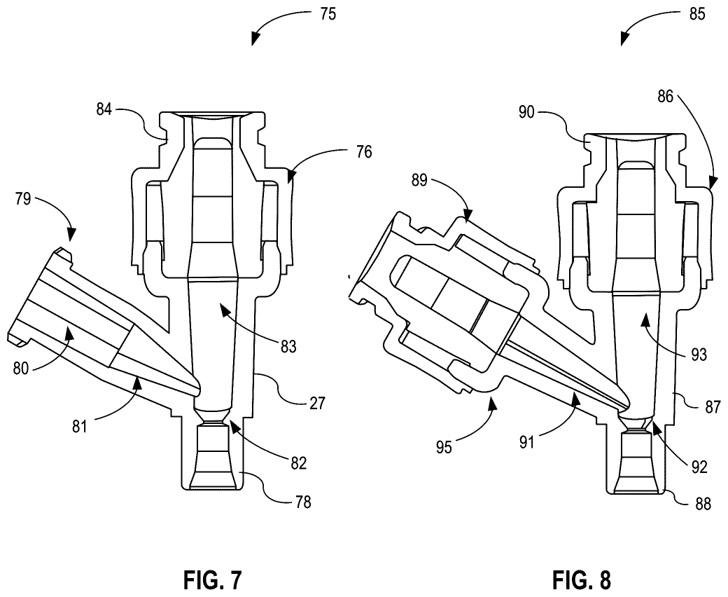

Next, referring to FIG. 7, a needle free connector 75 in accordance with another aspect of the present disclosure is shown. The needle free connector 75 includes a proximal body 76 and a distal body 77, with the proximal body 76 and the distal body 77 coupled together by any appropriate method. The proximal body 76 and distal body 77 define a flow path 83 extending between a distal port 78 and a proximal port 84.

The needle free connector 75 further includes a side port 79. In some embodiments, the side port 79 may be offset from a center of the flow path 83. Such an offset of the side port 79 may be configured to cause fluid entering the flow path 83 via the side port 79 to enter along an interior surface of the distal body 77 and cause a vortex or otherwise redirect fluid within the distal body 77 and the proximal body 76 to aid flushing of the needle free connector 75.

In some embodiments, the distal body 77 of the needle free connector 75 further includes internal structure 82 configured to create a vortex or otherwise redirect fluid when fluid enters the needle free connector 75 via the side port 79. In some embodiments, the offset and vortex-creating feature is the same or similar to the flushing features shown and described in U.S. Patent Application Publication No. 2021/0220548, which is hereby incorporated by reference in its entirety.

The proximal body 76 and the distal body 77 of the needle free connector 75 define a longitudinal axis extending between the distal port 78 and the proximal port 84, with the side port 79 extending from the distal body 77 at an angle of, e.g., 15°-165° relative to the longitudinal axis of the distal body 77.

Unlike the needle free connector 16 described above with respect to FIGS. 5 and 6, which included a side port configured to receive a length of extension tubing therein, the side port 79 of needle free connector 75 is configured as, e.g., a luer port to accommodate coupling to a removable connector. However, side port 79 does include a primary channel portion 80 and a tapered portion 81, wherein the tapered portion 81 provides fluid communication to the flow path 83. The tapered portion 81 acts to gradually reduce the inner diameter of the side port 79 between primary channel portion 80 and the flow path 83, acting as a nozzle to increase the fluid velocity of the fluid delivered through the side port 79 as it enters the flow path 83. As detailed above, such increased fluid velocity may aid in flushing performance to remove a substantial portion of blood remaining in the needle free connector 75 after a blood draw procedure.

Referring now to FIG. 8, a needle free connector 85 in accordance with another aspect of the present disclosure is shown. The needle free connector 85 includes a proximal body 86 and a distal body 87, with the proximal body 86 and the distal body 87 coupled together by any appropriate method. The proximal body 86 and distal body 87 define a flow path 93 extending between a distal port 88 and a proximal port 90.

The needle free connector 85 further includes a side port 95. In some embodiments, the side port 95 may be offset from a center of the flow path 93. Such an offset of the side port 95 may be configured to cause fluid entering the flow path 93 via the side port 95 to enter along an interior surface of the distal body 87 and cause a vortex or otherwise redirect fluid within the distal body 87 and the proximal body 86 to aid flushing of the needle free connector 85.

In some embodiments, the distal body 87 of the needle free connector 85 further includes internal structure 92 configured to create a vortex or otherwise redirect fluid when fluid enters the needle free connector 85 via the side port 95. In some embodiments, the offset and vortex-creating feature is the same or similar to the flushing features shown and described in U.S. Patent Application Publication No. 2021/0220548, which is hereby incorporated by reference in its entirety.

The proximal body 86 and the distal body 87 of the needle free connector 85 define a longitudinal axis extending between the distal port 88 and the proximal port 90, with the side port 95 extending from the distal body 87 at an angle of, e.g., 15°-165° relative to the longitudinal axis of the distal body 87.

Unlike the needle free connector 16 described above with respect to FIGS. 5 and 6, which included a side port configured to receive a length of extension tubing therein, the side port 95 of needle free connector 85 is configured to include a secondary needle free connector 89 to accommodate, e.g., a fluid injection device usable with a needle free interface. However, side port 95 includes a tapered portion 91, wherein the tapered portion 91 provides fluid communication to the flow path 93. The tapered portion 91 acts to gradually reduce the inner diameter of the side port 95, acting as a nozzle to increase the fluid velocity of the fluid delivered through the side port 95 as it enters the flow path 93. As detailed above, such increased fluid velocity may aid in flushing performance to remove a substantial portion of blood remaining in the needle free connector 85 after a blood draw procedure.

Although described with respect to needle free connectors used with integrated intravenous catheters, it is to be understood that the concepts described herein may be applicable to any medical device fluid junction having fluid inlets or outlets with different central axes. The fluid junction may be an optimization of one or more of side fluid path entrance angle, central or planar offset, side port flow directing ramp(s), and/or optimized position of proximal flow diverting feature(s).

Furthermore, it is to be understood that the needle free connectors described herein may be integrally provided with (and attached to) an integrated catheter system or, alternatively, the needle free connectors described herein may be utilized as a stand-alone extension set, provided separately from other components of an integrated catheter system.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. An integrated intravenous catheter system comprising:
a catheter adapter comprising a catheter and an inlet, the catheter configured to be inserted into a patient's vasculature;
a needle free connector comprising a proximal port, a distal port positioned opposite the proximal port, and a side port positioned between the proximal port and the distal port; and
extension tubing extending from the side port of the needle free connector,
wherein the side port of the needle free connector comprises a tube receiving portion having a first inner diameter sized and configured to receive a distal portion of the extension tubing, an inlet portion having a second inner diameter smaller than the first inner diameter, and a tapered portion extending between the tube receiving portion and the inlet portion.

2. The integrated intravenous catheter system of claim 1, wherein the needle free connector comprises a first body portion and a second body portion, wherein the first body portion and the second body portion define a flow path extending between the proximal port and the distal port.

3. The integrated intravenous catheter system of claim 2, wherein the inlet portion of the side port fluidly couples the extension tubing to the flow path of the needle free connector.

4. The integrated intravenous catheter system of claim 2, wherein the side port is offset from a center of the flow path.

5. The integrated intravenous catheter system of claim 2, wherein the second body portion of the needle free connector comprises an internal structure configured to redirect fluid when fluid enters the needle free connector via the side port.

6. The integrated intravenous catheter system of claim 2, wherein the first body portion and the second body portion of the needle free connector define a longitudinal axis extending between the proximal port and the distal port, and wherein the side port extends from second body portion at an angle of 30-150 degrees relative to the longitudinal axis.

7. The integrated intravenous catheter system of claim 1, wherein an interior diameter of the extension tubing is greater than the second inner diameter of the inlet portion of the side port.

8. The integrated intravenous catheter system of claim 1, wherein the proximal port of the needle free connector comprises a valve member.

9. The integrated intravenous catheter system of claim 8, wherein the valve member comprises a split septum valve.

10. The integrated intravenous catheter system of claim 1, further comprising a medical connector positioned at a proximal end of the extension tubing.

11. A needle free connector comprising:
a first body portion;
a second body portion coupled to the first body portion;
a proximal port positioned at a proximal end portion of the first body portion;
a distal port positioned at a distal end portion of the second body portion; and
a side port positioned between the proximal port and the distal port,
wherein the side port comprises a tube receiving portion having a first inner diameter, an inlet portion having a second inner diameter smaller than the first inner diameter, and a tapered portion extending between the tube receiving portion and the inlet portion.

12. The needle free connector of claim 11, wherein the first body portion and the second body portion define a flow path extending between the proximal port and the distal port.

13. The needle free connector of claim 12, wherein the side port is offset from a center of the flow path.

14. The needle free connector of claim 11, wherein the second body portion comprises an internal structure configured to redirect fluid when fluid enters the needle free connector via the side port.

15. The needle free connector of claim 11, wherein the first body portion and the second body portion of the needle free connector define a longitudinal axis extending between the proximal port and the distal port, and wherein the side port extends from second body portion at an angle of 30-150 degrees relative to the longitudinal axis.

16. The needle free connector of claim 11, wherein the proximal port comprises a valve member.

17. The needle free connector of claim 16, wherein the valve member comprises a split septum valve.

18. A needle free connector comprising:
a first body portion;
a second body portion coupled to the first body portion;
a proximal port positioned at a proximal end portion of the first body portion;
a distal port positioned at a distal end portion of the second body portion, wherein the first body portion and the second body portion define a flow path extending between the proximal port and the distal port; and
a side port positioned between the proximal port and the distal port,
wherein the side port comprises a primary channel portion and a tapered portion extending between the primary channel portion and the flow path.

19. The needle free connector of claim 18, wherein the side port comprises a luer connector on a proximal end thereof.

20. The needle free connector of claim 18, wherein the side port comprises a secondary needle free connector positioned on a proximal end thereof.
